(19) United States
(12) Patent Application Publication
(10) Pub. No.: US 2020/0345044 A1
(43) Pub. Date: Nov. 5, 2020

(54) HIGHLY ACTIVE ANTIOXIDANT BASED ON TREHALULOSE

(71) Applicant: Suedzucker Aktiengesellschaft Mannheim/Ochsenfurt, Mannheim (DE)

(72) Inventors: Joerg Kowalczyk, Eisenberg-Steinborn (DE); Stephan Hausmanns, Mannheim (DE); Tillmann Doerr, Hohen-Suelzen (DE)

(21) Appl. No.: 16/931,697

(22) Filed: Jul. 17, 2020

Related U.S. Application Data
(63) Continuation of application No. 14/605,295, filed on Jan. 26, 2015, now abandoned.
Application No. 14/605,295 is a division of application No. 12/997,547, filed on Dec. 10, 2010, now abandoned.

(30) Foreign Application Priority Data
Jun. 11, 2008 (DE) 10 2008 027 686.3
Nov. 25, 2008 (DE) 10 2008 060 009.1

(51) Int. Cl. A23L 3/3562; A23C 9/13; C12C 12/02; A61K 8/60; A61K 31/7016; C12C 5/02; A61Q 19/00; A23K 20/163; A23K 20/158; A23L 33/12; A23L 33/10; A23L 27/30; A23C 3/08; A23L 2/44; C12C 11/11

(52) U.S. Cl. CPC A23L 3/3562; A61K 2800/522; A23C 9/1315; C12C 12/02; A61K 8/60; A61K 31/7016; C12C 5/02; A61Q 19/00; A23K 20/163; A23K 20/158; A23L 33/12; A23L 33/10; A23L 27/33; A23C 3/08; A23L 2/44; C12C 11/11; A23V 2002/00; A23C 9/1307

(57) ABSTRACT

The invention provides an improved antioxidant for food, animal feed, cosmetics and pharmaceuticals, as well as compositions that contain this antioxidant as preferably the only additive having an anitoxidative effect.

This is a continuation of U.S. application Ser. No. 14/605,295, filed Jan. 26, 2015, which is a divisional of U.S. application Ser. No. 12/997,547, filed on Dec. 10, 2010, now abandoned, which is a national stage entry of PCT/EP2009/002599, filed Apr. 8, 2009, the entire contents of which are incorporated herein by references in their entireties.

The invention relates to the technical field of additives or adjuvants for food, animal feed, cosmetics and pharmaceuticals, in particular, to additives and antioxidants with an antioxidative effect. The present invention primarily provides an improved antioxidant for food, animal feed, cosmetics and pharmaceuticals, as well as food, animal feed, cosmetics and pharmaceuticals that contain this antioxidant as preferably the only anitoxidative additive.

Additives or adjuvants with an antioxidative effect for food, pharmaceuticals or cosmetics are known. They primarily suppress the appearance of decomposition products that arise in the manufacturing or storage of food, animal feed, cosmetics or pharmaceuticals when oxidation-sensitive active ingredients contact atmospheric oxygen or other oxidative substances. Food will be referred to below in such a manner that it is understood to mean not only preferably food and feed, but also any other agents and compositions, such as cosmetics or pharmaceuticals, that could be applied in and on an animal or human body. As food additives, antioxidants have the effect of improving the shelf life or aging stability of the food. They also function as a therapeutic and/or prophylactic active ingredient that takes effect in the animal or human body and suppresses harmful oxidative processes there. One example of such an antioxidant is ascorbic acid (vitamin C). Ascorbic acid and its salts are added, for example, to fruit drinks, preserves, condensed milk or meat products.

Antioxidant agents are food additives or adjuvants that are generally present in a much lower concentration compared to an oxidation-sensitive substrate and can markedly slow or prevent its oxidation. In addition to their property of being reduction agents themselves and thus a substrate for oxidative processes, antioxidants can be distinguished as binding metal ions, such as divalent iron, having a catalytic action in oxidation reactions in the form of chelates and/or suppressing radical chain reactions by capturing the starter radical (scavenging or quenching) or by capturing an intermediate radical (chain-breaking).

Many antioxidants, including ascorbic acid, have a sometimes considerable taste-altering effect on food. This effect can be used deliberately. Thus, ascorbic acid has a sour taste resembling lemon juice. In general the antioxidative effect of the antioxidant is dependent on the pH value. Thus ascorbic acid has a strong antioxidative effect only in acids. In many foods, however, the sour taste is undesirable, or it is not possible to maintain an acidic environment, for example, in fresh milk products. Harmful health effects of some well-known antioxidants on the animal or human body have now been discovered. This also includes ascorbic acid, as well as sulfur dioxide and its salts (sulfite, bisulfite, disulfite, hydrogen sulfite).

In addition, many of the known antioxidants have an excessively strong reducing effect and therefore lead under certain conditions to undesired reactions with components of the food.

Many known antioxidants, such as ascorbic acid, are themselves very sensitive to oxidation or light, which makes it more difficult to use them industrially.

There is therefore a need to make additional substances available for use as antioxidants that have an antioxidative effect without the disadvantages of known antioxidants.

Another aspect of the invention relates to brewing technology, particularly the production of beer with long shelf life and agents therefor. Beer is known to be a food with an unstable taste, which is subject to a natural aging process. The taste stability represents an important quality feature of a beer intended for storage. The general goal that is pursued is to retain the original character of the beer from bottling to consumption. The aging process is characterized primarily by the oxidative decomposition of components in the beer and by the so-called “aging components” that result. The latter change the taste of the beer disadvantageously. The entry of oxygen after fermentation and during bottling is essential to the oxidative decomposition. The molecular atmospheric air forms reactive types of oxygen, above all the hydroxyl radical. The hydroxyl radical primarily oxidizes the ethanol, free fatty acids and isohumulone components present in the beer into aldehydes and ketones; the hydroxyl radical also serves as a starter radical for reactions into additional radical forms, from which aldehydes again arise.

Beer naturally contains a number of components with a reducing effect, preventing the formation of such disadvantageous oxidation products over a certain period of time. This so-called endogenous antioxidative activity or the endogenous antioxidative potential (EAP) of the beer provides a beer with a certain storage stability. Components with an antioxidative effect contained in beer are above all sulfur dioxide, free phenols, polyphenols or xanthohumols. If the antioxidative capacity of these components is exhausted, then the end of the beer's shelf life has been reached. By nature, beer therefore has only a limited shelf life on its own.

Sulfur dioxide is in part formed during the main fermentation by the brewing yeast being used. The formation of sulfur dioxide, however, is dependent on the course of the fermentation process and the strain of the yeast being used. If the endogenous antioxidant potential of the beer is to be increased by sulfur dioxide, a special fermentation course and a special selection of yeast must be made; the flexibility in the practice of brewing is restricted.

Various technological measures during the production of beer have been used to increase the proportions of such phenolic substances in the beer. Such methods are in part expensive and change the brewing result; furthermore the flexibility in the practice of brewing is restricted.

It is well known that sulfur dioxide is an antioxidant added to beer. It is generally added to beers intended for longer storage such as those for export overseas. Sulfur dioxide disadvantageously changes the taste of the beer from the very beginning. Other known antioxidants such as ascorbic acid likewise change the taste of beer disadvantageously.

A problem of the present invention is therefore also to provide agents to increase the endogenous antioxidant potential (EAP) of beer, mixed beer beverages and similar brewery products without having to accept taste disadvantages and the other known disadvantages associated with antioxidants.

The technical problem underlying the invention is solved by the provision of an antioxidant or an antioxidant composition preferably for use in food, animal feed, cosmetics and pharmaceuticals that contains trehalulose or trehalulose-containing syrup as the component with an antioxidative effect. The antioxidant according to the invention preferably contains trehalulose or trehalulose-containing syrup as the only component with an antioxidative effect or consists thereof. In one variant of the invention, trehalulose or trehalulose-containing syrup is a component of an antioxidant composition which contains, in addition to trehalulose, at least one additional component that synergistically supports the antioxidative effect of trehalulose or trehalulose-containing syrup. Such a synergistic effect is preferably combined with the complexing or chelation of oxidative, catalytically active metal ions.

The inventors surprisingly found that the addition of trehalulose or trehalulose-containing syrup markedly improves the oxidative stability of food or animal feed. Trehalulose acts as an adjuvant for efficient improvement of the shelf life, aging stability or oxidation stability. Trehalulose effectively prevents or reduces the appearance of oxidation decomposition products, so-called aging components which, in food such as beer or similar beverages, limit the shelf life due to their effects that can negatively affect taste and/or can be hazardous to health. Trehalulose has a protective effect for oxidation-sensitive food components such as colorants, artificial flavors and pharmaceutically active ingredients, and saturated fatty acids including above all omega-3 and/or omega-6 fatty acids and comparable fatty acids. Without being bound by theory, trehalulose surprisingly shows a remarkably greater antioxidative effect, which is therefore efficiently usable, as compared to other known reducing sugars such as glucose.

The invention therefore provides for the use of the substance trehalulose as an antioxidant for food, cosmetic products and pharmaceutical preparations. Trehalulose is preferably used as the only antioxidant added to the product.

Trehalulose, alpha-D-glucopyranosyl-1,1-beta-D-fructofuranose is a disaccharide of glucose and fructose and a structural isomer of saccharose. It is primarily obtained by isomerizing saccharose. Trehalulose-containing syrup, also referred to as trehalulose syrup, is preferably obtained from saccharose in a biocatalytic process using immobilized and non-reproductive cells from Pseudomonas mesoacidophila, the strain MX-45 for instance, or corresponding other transgenic organisms, preferably at a temperature of 10 to 10° C. in a conventional manner. The obtained raw syrup contains trehalulose in a proportion of 75-92 wt %, more frequently in a proportion of 85-90% (weight of the dry substance). It can be used directly as trehalulose-containing syrup in accordance with the invention. The proportion of trehalulose can be increased, and the proportion of foreign substances and residual saccharose can be decreased, by known methods such as chromatography and crystallization.

Trehalulose-containing syrups according to the invention typically have the following composition:

| | Standard % (HPLC-NH2) | g/100 g TS (GC) |
|---|---|---|
| Trehalulose | 89.3-89.5 | 88.9 |
| Fructose | 0.2 | 0.32 |
| Glucose | 0.2-0.3 | 0.38 |
| Saccharose | 0.3-0.4 | 0.17 |
| Isomaltulose | 6.8 | 11 |
| Isomaltose | 0.2-0.3 | 0 |
| DP-3 | <0.1 | |
| Isomelezitose | <0.1 | |
| Rest | 2.7 | 0.94 |

It is understood that this composition can differ and fluctuate within a certain range. Trehalulose syrups according to the invention may contain leucrose in a proportion of roughly 1%. Trehalulose syrups may also contain residual saccharose in a proportion of roughly 1%.

The invention is not limited to these trehalulose syrups. In connection with this invention, trehalulose syrup is understood to mean a trehalulose-containing composition containing at least 60 wt % trehalulose, but preferably 70 wt %, or more preferably 80 wt % or more.

In the preferred embodiments, the proportion of isomaltulose is lower and is always less than 15% and if needed less than 5%. Isomaltulose can cause the appearance of crystallization in the end product; this may not be desired depending on the end product. No isomaltulose is contained in other preferred embodiments. Such variants can be obtained by crystallization of the byproducts, above all isomaltulose.

Trehalulose and trehalulose-containing syrups were previously intended primarily as a replacement of saccharose in certain saccharose-containing food formulations. Trehalulose was used above all as a sweetener to provide body (“bulk substance”). The present invention, on the other hand, provides a different teaching. Trehalulose or trehalulose-containing syrups are used as antioxidative adjuvants or additives (antioxidants) particularly for food and feed, but also for cosmetic and pharmaceutical compositions. The invention thus lies in a different technical field of use. According to the invention, trehalulose can also be used in compositions and food in which the sweetening effect and/or the function as a body-providing component is not necessary and/or does not appear. The use of trehalulose or trehalulose-containing syrups according to the invention thus also lies outside the field of sweets and/or outside that of carbohydrate-containing food. This includes, for example, protein-rich and/or fat-rich food such as dairy products (cheese, yogurt, etc.) or preparations containing oils and fats (margarines, food oils, etc.), as well as animal feed with a high content of oxidation-sensitive fatty acids, for example, those for milk production.

Is understood that the presence of other substances with an antioxidative effect in a trehalulose composition according to the invention can support the antioxidative or protective effect of trehalulose.

The inventors surprisingly found that the presence of isomaltulose in the trehalulose composition superadditively or synergistically improves the antioxidative or protective effect of trehalulose. A critically preferred embodiment of the invention is the use of a composition containing isomaltulose and trehalulose as preferably the only component with an antioxidative or protective effect in food, animal feed, cosmetics or pharmaceuticals. In one variant of this embodiment, the content of trehalulose in this composition is at least 50 wt % or more and the content of isomaltulose is 50 wt % or less. In another variant, the trehalulose is the component that superadditively improves the antioxidative or protective effect of isomaltulose. In this variant, the content of trehalulose in this composition is 50 wt % or less and the content of isomaltulose is 50 wt % or more. Accordingly, the subject matter of the invention also includes the use of trehalulose or trehalulose-containing syrup for improving/enhancing the antioxidative or protective effect of isomaltulose or isomaltulose-containing compositions.

The invention also relates to food, cosmetic products and pharmaceutical preparations that contain the antioxidant according to the invention. The invention also relates to the use of trehalulose or trehalulose-containing syrups as an antioxidant in such a product. The product preferably contains trehalulose or trehalulose-containing syrup as the only antioxidant added to the product. It is not out of the question for at least one additional component to be added to the trehalulose to support or increase the antioxidative effect of trehalulose, preferably in connection with a synergistic mode of action.

A food according to the invention that contains trehalulose or trehalulose-containing syrup as an antioxidant is preferably selected from:

i. milk products and dairy products such as cheese, butter, yogurt, kefir, quark [a type of fresh cheese], sour milk, buttermilk, cream, condensed milk, powdered milk, whey, lactose, milk protein, mixed-milk, low-fat milk, mixed-whey or butterfat products or preparations;

ii. pudding, crème, mousse and other desserts;

iii. butterfat products, mixed fat products, edible fats and edible oils;

iv. baked goods such as bread, including pastries and specialty baked goods, long-life cookies and cakes, biscuit products and wafers;

v. bread spreads, in particular fat-containing bread spreads, margarine products and shortenings;

vi. instant products and steeped beverages;

vii. fruit products or preparations such as preserves, marmalades, jellies, fruit compote, fruit pulps, fruit concentrate, fruit juices, fruit-juice concentrates, fruit nectar and powdered fruit juice;

viii. cereals, muesli and cereal mixtures as well as finished cereal-containing products such as muesli bars and breakfast products;

ix. primary nonalcoholic beverages, beverage bases and beverage powders, chocolate drinks, chocolate-drink powders;

x. primary alcoholic drinks and fermented products, wine, mixed wine beverages, beer, mixed beer beverages, alcohol-free beer or mixed beer beverage, reduced-alcohol beer or mixed beer beverage;

xi. meat products and sausage products;

xii. sweets such as chocolates, hard caramels, soft caramels, chewing gum, drops, fondant products, jelly products, licorices; foamed sweets, flakes, drops, compressed sweets, candied fruits, pralines, nougat products, ice-cream confections, marzipan and ice cream.

Of course the invention also relates to food and animal feed that are derived from the above-mentioned products, in particular special dietetic food. The invention further pertains to foods that are not conceived of or suitable for human consumption, or not exclusively so; these include food, animal feed, precursor mixtures for animal feed, high-starch animal feed, high-protein animal feed, high-fat animal feed, high-energy feed and high-energy feed concentrate. It was surprisingly found that the original content of oxidation-sensitive fatty acids in the animal feed, for example, omega-3 fatty acids in forage or forage preparations, or the content of added fatty acids can be preserved due to the protective/antioxidative action of trehalulose or trehalulose syrup, and not only during the storage of the feed, but also during the digestion/utilization of the feed in the animal. Oxidation-sensitive nutritional components or additives are thus available to the animal in a larger amount.

The target of the invented application of trehalulose or trehalulose-containing syrups as an antioxidant is food containing at least one component which is oxidation-sensitive and is subject to aging processes, which would reduce the freshness period or shelf life of the food. This is to be understood according to the invention as substances or substance mixtures that are subjected to oxidative decomposition during the manufacturing or storage of the food. This oxidative decomposition is preferentially initiated by contact with oxygen-containing components, above all by contact with atmospheric oxygen. The oxidative decomposition can also be caused by additional substances having an oxidative effect on their own that are contained in the food or a food composition. These include, for example, oxidizing acids, metals in a high oxidation state and their compounds, preservatives with an oxidative effect, as well as other oxygen, sulfur or halogen compounds with an oxidative effect. Trehalulose or trehalulose-containing syrup as an antioxidant also increases the stability in such products against free radicals and suppresses the formation of free radicals.

A preferred subject matter of the invention is a food that contains trehalulose or trehalulose-containing syrup as preferably the only antioxidant and contains a milk product or mixed milk product, above all a yogurt, and contains unsaturated fatty acids, above all omega-3 fatty acids, omega-6 fatty acids and/or similar ones as an oxidation-sensitive component. It has been surprisingly found that trehalulose as an antioxidant effectively suppresses the oxidative decomposition of omega-3 and omega-6 fatty acids. Added omega-3 fatty acid, omega-6 fatty acid or similar oxidation-sensitive component (see above) in a milk product, especially a yogurt, that contains trehalulose is degraded only to a slight extent even in long-term storage.

Another preferred subject matter of the invention is beer or modified forms thereof such as mixed beer beverage or alcohol-free or reduced-alcohol beer or mixed beer beverage, wherein trehalulose or trehalulose-containing syrup is contained as preferably the only antioxidant, especially preferably the only added one. It has surprisingly been shown that a trehalulose-containing beer has a particularly long shelf life. Even in long-term storage, the aging-induced disadvantageous taste changes remain within tolerable limits. Trehalulose stabilizes oxidation-sensitive components of the beer and increases the EAP value. Trehalulose prevents early accumulation of the aging components that are responsible for the so-called “aged taste” of stored beer.

Subject matter of the invention is therefore also the use of trehalulose or trehalulose-containing syrup for improving the aging stability, oxygen stability and/or shelf life of food, animal feed, cosmetics and pharmaceuticals, particularly of oxidation-sensitive food, animal feed, cosmetics and pharmaceuticals, in particular beer, mixed beer beverages, instant beverages and chocolate instant beverages; above all the use of trehalulose or trehalulose-containing syrup for reducing the appearance of aging components that negatively affect taste in beer or mixed beer beverages. One subject matter of the invention is finally also the use of trehalulose for reducing the oxidation of oxidation-sensitive colorants, flavorings, pharmaceutical active ingredients and/or unsaturated fatty acids, above all omega-3 fatty acids and the like in food, particularly in yogurt or the like.

The invention will be illustrated in detail based on examples and figures, without these being understood as limiting.

The figures show:

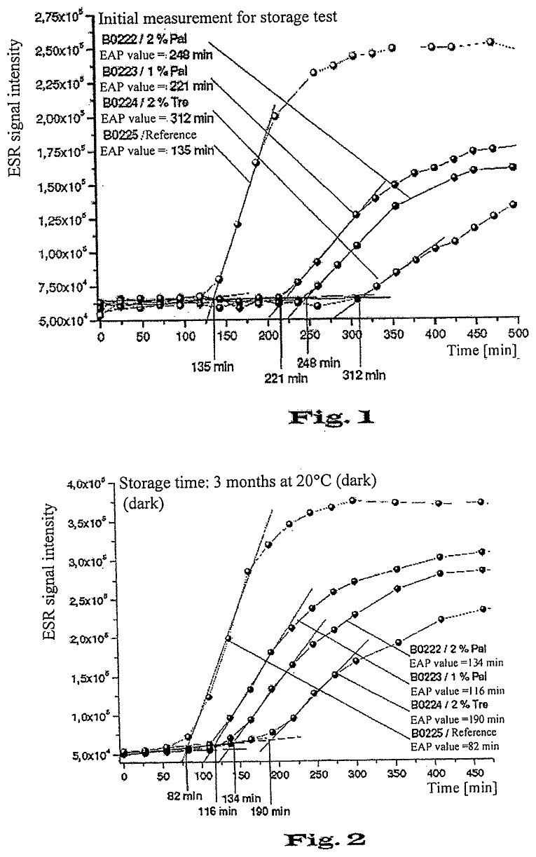

FIG. 1: ESR measurement (signal intensity and EAP value) of freshly produced batches of diet beer with the addition of trehalulose, isomaltulose or without additives.

FIG. 2: ESR measurement (signal intensity and EAP value) of the aged batches (see above) after three months of storage at 20° C. in the dark.

### EXAMPLE 1: STABILIZATION OF OMEGA-3 FATTY ACIDS IN MILK PRODUCTS

The ability of trehalulose-containing syrup (ca. 89 wt % trehalulose) to suppress or reduce the oxidative decomposition of omega-3 fatty acids embedded in a yogurt matrix was studied.

For this purpose, whole-milk yogurt (yogurt, mild, 3.5% fat, Milram brand) was used, into which DHA CL (Lonza) was stirred as omega-3 fatty acid. The following yogurt preparations (g per 100 g) were produced:

| | |
|---|---|
| Batch A (according to the invention): | 5 g trehalulose syrup |
| Batch B: | 5 g fructose |
| Batch C: | 5 g saccharose |

150 mg DHA-CL (Lonza) and roughly 40 mg of a saturated fatty acid (C22:0) as an internal standard were stirred into 240 g of each of these batches by means of an Ultraturrax mixer. All measures took place under a nitrogen protective atmosphere.

Immediately after production of each of the yogurt preparations, respective control samples were drawn and the recovery rate of omega-3 fatty acids in the freshly produced preparations was determined.

To determine the omega-3 fatty acids, 0.85 mL of the yogurt preparation was pipetted into a test tube (Eppendorf), weighed, mixed with 1 mL tert-butyl methyl ether and intensively agitated. After roughly 3 min of agitation, the test tubes were centrifuged at 13,000 rpm for 3 min. Then 200 μL of the clear supernatant for each sample were removed and respectively mixed with 100 μL THMS. 1 μL of each solution was injected into a gas chromatograph (Agilent, model 6890) that was optimized in a conventional manner for the detection of the fatty acids being used.

Recovery rates of 96-99% of the originally used omega-3 fatty acids were found without a difference in all of the control samples.

The yogurt preparations produced were then stored for 28 days at 7° C. The samples were each analyzed by gas chromatography.

Results: The trehalulose-containing preparations (batch A) showed the highest recovery rates for the oxidation-sensitive fatty acids. The taste of these preparations is unobjectionable. The samples with added fructose or saccharose displayed considerably lower recovery rates. Impaired tastes appear (fatty taste).

It surprisingly was shown that fructose, which acts as a reducing sugar by means of the keto-en(di)ol tautometry, has a markedly lower protective effect than trehalulose, even at high molarity. Without being bound to any theory, the surprisingly high antioxidative effect of trehalulose is not solely attributable to the presence of reactive aldehyde groups. The reducing activity (redox potential) of aldehyde sugars was generally considered too low for the observed strong antioxidative effect to be derived from it alone. Other mechanisms appear to support the antioxidative and protective effect in this case.

### EXAMPLE 2: STABILIZATION OF BEER

To check the influence on the oxidation stability of beer, trehalulose was added (according to the invention) to commercial diet beer (containing almost no carbohydrates). In comparison samples, isomaltulose (comparison) was added to commercial diet beer, or the diet beer was used without an additive. All samples were subjected to defined aging of the beer. The oxidation stability was measured by means of ESR at the beginning and the end of the beer aging.

Commercial Diet Beer

| | |
|---|---|
| Wort [measured %] | 9.1 |
| Apparent residual extract [wt %] | 0 |
| Actual residual extract [wt %] | 1.66 |
| Alcohol content [vol %] | 4.78 |
| Bitterness units [BU] | 24 |

Batch 1: Diet beer with 2 g/100 mL trehalulose syrup (according to the invention)

Batch 2: Diet beer with 1 g/100 mL isomaltulose (comparative example)

Batch 3: Diet beer with 2 g/100 mL isomaltulose (comparative example)

Batch 4: Diet beer without additives (comparative example)

All batches were stored for 3 months at 20° C. in the dark (beer aging test).

The oxidative stability of the sample was determined by means of ESR (electron spin resonance spectroscopy) according to the methodology of Methner and Kunz (Methner and Kunz, 2006: more accurate prognoses of oxidative beer stability by means of ESR spectroscopy Brauerei Forum 2006: 7-9). A higher EAP value in this case indicates a more favorable oxidative stability. The absolute signal intensity continues to be used for assessment, since it is a measurement of the radical substances formed by oxidative processes.

FIG. 1 shows the results of the ESR measurements (signal intensity and EAP value) of the freshly manufactured batches (see above) after cold storage (0-2° C.).

FIG. 2 shows the results of the ESR measurement (signal intensity and EAP value) of the aged batches (see above) after 3 months storage at 20° C. in the dark.

The EAP value for batch 1 with trehalulose according to the invention (“2% Tre”) is the highest: EAP=312 min in the fresh batch and EAP=190 min in the 3-month aged batch. The ESR signal intensity for batch 1 according to the invention (“2% Tre”) is the lowest. This indicates the high antioxidative potential of trehalulose as an agent with an antioxidative effect.

In comparison, isomaltulose (batches 2 and 3; “1% Pal” and “2% Pal”) does show an antioxidative effect as well. For an identical added amount, however, it is much less pronounced. The invention provides an agent with an antioxidative effect (antioxidant) in the form of trehalulose, which surprisingly is much more strongly antioxidative than other saccharides, and in particular, other low-glycemic saccharides, above all the disaccharide isomaltulose.

1. A method for improving the aging stability, the oxidation stability and/or shelf-life of an oxidation-sensitive food, animal feed, cosmetic or pharmaceutical composition containing at least one oxidation-sensitive component comprising incorporating a trehalulose-containing composition containing an antioxidant effective amount of trehalulose and a synergistically effective amount isomaltulose into the oxidation-sensitive food, animal feed, cosmetic or pharmaceutical composition, wherein the content of trehalulose in the composition is at least 50 wt.-% or more and the content of isomaltulose is 50 wt.-% or less.

2. The method according to claim 1, wherein the at least one oxidation-sensitive component is an oxidation-sensitive unsaturated fatty acid.

3. The method according to claim 2, wherein the unsaturated fatty acid is selected from the group consisting of omega-3 fatty acids and omega-6 fatty acids.

4. The method according to claim 1, wherein the food is selected from the group consisting of: i. milk products and dairy products; ii. pudding, crème, mousse and other desserts; iii. butterfat products, mixed fat products, edible fats and edible oils; iv. baked goods; v. bread spreads; vi. instant products and steeped beverages; vii. fruit products or preparations; viii. cereals, muesli and cereal mixtures, muesli bars and breakfast products; ix. primary nonalcoholic beverages, beverage bases and beverage powders, chocolate drinks, chocolate-drink powders; x. primary alcoholic drinks and fermented products, wine, mixed wine beverages, beer, mixed beer beverages, alcohol-free beer or mixed beer beverage, reduced-alcohol beer or mixed beer beverage; xi. meat products and sausage products; xii. sweets; and dietetic nutritional products derived therefrom.

5. The method according to claim 1, wherein the animal feed is an animal food concentrate.

6. The method according to claim 1, wherein the food is a milk product, yogurt or mixed milk product containing omega-3 or omega-6 fatty acid.

7. The method according to claim 1, wherein the food is a beer, alcohol-free beer or reduced-alcohol beer.

8. The method according to claim 1, wherein the trehalulose and the isomaltulose are incorporated in form of a single syrup.

9. The method according to claim 8, wherein the syrup is the only adjuvant with an antioxidative effect.

10. The method according to claim 9, wherein the at least one oxidation-sensitive component is at least one oxidation-sensitive unsaturated fatty acid.

11. The method according to claim 10, wherein the unsaturated fatty acid is selected from the group consisting of omega-3 fatty acids and omega-6 fatty acids.

12. The method according to claim 11, wherein the food is selected from the group consisting of: i. milk products and dairy products; ii. pudding, créme, mousse and other desserts; iii. butterfat products, mixed fat products, edible fats and edible oils; iv. baked goods; v. bread spreads; vi. instant products and steeped beverages; vii. fruit products or preparations; viii. cereals, muesli and cereal mixtures, muesli bars and breakfast products; ix. primary nonalcoholic beverages, beverage bases and beverage powders, chocolate drinks, chocolate-drink powders; x. primary alcoholic drinks and fermented products, wine, mixed wine beverages, beer, mixed beer beverages, alcohol-free beer or mixed beer beverage, reduced-alcohol beer or mixed beer beverage; xi. meat products and sausage products; xii. sweets; and dietetic nutritional products derived therefrom.

13. The method according to claim 12, in which: the milk products and dairy products are selected from the group consisting of cheese, butter, yogurt, kefir, quark, sour milk, buttermilk, cream, condensed milk, powdered milk, whey, lactose, milk protein, mixed milk, low-fat milk, mixed-whey or butterfat preparations; the baked goods are selected from the group consisting of bread, pastries and specialty baked goods, long-life cookies and cakes, biscuit products and wafers; the bread spreads are selected from the group consisting of fat-containing bread spreads, margarine products and shortenings; the fruit products or preparations are selected from the group consisting of preserves, marmalades, jellies, fruit compote, fruit pulps, fruit concentrate, fruit juices, fruit-juice concentrates, fruit nectar and powdered fruit juice; and the sweets are selected from the group consisting of chocolates, hard caramels, soft caramels, chewing gum, drops, fondant products, jelly products, licorices; foamed sweets, flakes, drops, compressed sweets, candied fruits, pralines, nougat products, ice-cream confections, marzipan and ice cream.

14. The method according to claim 13, wherein the food is a milk product, yogurt or mixed milk product containing omega-3 or omega-6 fatty acid.

15. The method according to claim 14, wherein the food is a beer, alcohol-free beer or reduced-alcohol beer.

16. The method according to claim 4, in which: the milk products and dairy products are selected from the group consisting of cheese, butter, yogurt, kefir, quark, sour milk, buttermilk, cream, condensed milk, powdered milk, whey, lactose, milk protein, mixed milk, low-fat milk, mixed-whey or butterfat preparations; the baked goods are selected from the group consisting of bread, pastries and specialty baked goods, long-life cookies and cakes, biscuit products and wafers; the bread spreads are selected from the group consisting of fat-containing bread spreads, margarine products and shortenings; the fruit products or preparations are selected from the group consisting of preserves, marmalades, jellies, fruit compote, fruit pulps, fruit concentrate, fruit juices, fruit-juice concentrates, fruit nectar and powdered fruit juice; and the sweets are selected from the group consisting of chocolates, hard caramels, soft caramels, chewing gum, drops, fondant products, jelly products, licorices; foamed sweets, flakes, drops, compressed sweets, candied fruits, pralines, nougat products, ice-cream confections, marzipan and ice cream.

17. The method according to claim 16, wherein the food is a milk product, yogurt or mixed milk product containing omega-3 or omega-6 fatty acid.

18. The method according to claim 17, wherein the food is a beer, alcohol-free beer or reduced-alcohol beer.
